(19) United States
(12) Patent Application Publication
(10) Pub. No.: US 2022/0002363 A1
(43) Pub. Date: Jan. 6, 2022

(54) COMPOSITIONS AND METHODS FOR TREATING PULMONARY DISEASE

(71) Applicants: United States Government As Represented By The Department of Veterans Affairs, Washington, DC (US); University of Washington, Seattle, WA (US)

(72) Inventors: Gustavo Matute-Bello, Seattle, WA (US); Osamu Kajikawa, Seattle, WA (US)

(21) Appl. No.: 17/366,844

(22) Filed: Jul. 2, 2021

Related U.S. Application Data
(60) Provisional application No. 63/047,932, filed on Jul. 3, 2020.

(51) Int. Cl. C07K 14/47; A61P 11/00

(52) U.S. Cl. CPC C07K 14/4703; A61K 38/00; A61P 11/00

(57) ABSTRACT

Disclosed herein, are peptides capable of inhibiting sFasL activity, and pharmaceutical compositions containing the peptides and methods treating a pulmonary disease.

### CROSS REFERENCE TO RELATED APPLICATIONS

This application claims the benefit of U.S. Provisional Application No. 63/047,932, filed Jul. 3, 2020. The content of this earlier filed application is hereby incorporated by reference herein in their entirety.

### GOVERNMENT SUPPORT

This invention was made with government support under Grant Number HL127075-05 awarded by the National Institutes of Health and Grant Number BX002914 awarded by the United States Department of Veterans Affairs Biomedical Laboratory R&D Service. The government has certain rights in this invention.

### INCORPORATION OF THE SEQUENCE LISTING

The present application contains a sequence listing that is submitted via EFS-Web concurrent with the filing of this application, containing the file name “37759 0236U3 SL.txt” which is 8,192 bytes in size, created on Jul. 2, 2021, and is herein incorporated by reference in its entirety.

### BACKGROUND

The acute respiratory distress syndrome (ARDS) is defined by the sudden onset of bilateral lung infiltrates and impaired gas exchange, in the absence of evidence of left ventricular dysfunction (Bernard G A, et al. Consensus Committee. J Crit Care 9: 72-81, 1994; and Ranieri V M, et al. JAMA 307: 2526-2533, 2012). ARDS is an important clinical problem in the United States, affecting 200,000 patients per year and resulting in death of approximately 75,000 persons (Rubenfeld G D, et al. N Engl J Med 353: 1685-1693, 2005). Over the past 20 years the mortality due to ARDS has decreased from approximately 60% to 30-40%, in part because of the discovery that mechanical ventilation with large tidal volumes is deleterious; however, mortality remains unacceptably high (Li G, et al. Am J Respir Crit Care Med 183: 59-66, 2011). Furthermore, there is increasing evidence that survivors suffer significant long-term consequences (Herridge M S, et al. Intensive Care Med 42: 725-738, 2016). Despite these negative outcomes, specific treatments are lacking.

### SUMMARY

Described herein are compositions comprising a peptide comprising the amino acid sequence QLFX₁LQX₂X₃LAX₄LX₅X₆STSQMX₇TASSLX₈K, wherein X₁ is H or a non-polar amino acid residue; X₂ is K or a non-polar amino acid residue; X₃ is E or a non-polar amino acid residue; X₄ is E or a non-polar amino acid residue; X₅ is R or a non-polar amino acid residue; X₆ is E or a non-polar amino acid residue; X₇ is H or a non-polar amino acid residue; and X₈ is E or a non-polar amino acid residue.

Disclosed herein are compositions comprising a peptide comprising the amino acid sequence of SEQ ID NO: 1: QLFHLQKELAELRESTSQMHTASSLEK, wherein one or more of the charged amino acids in SEQ ID NO: 1 is substituted for a non-polar amino acid residue.

Disclosed herein are compositions comprising a recombinant sFAsL polypeptide, wherein one or more of the charged amino acid residues in the stalk region is substituted for a non-polar amino acid residue.

### BRIEF DESCRIPTION OF THE DRAWINGS

FIG. 1 shows the bioactivity of the sFasL mutant protein. Jurkat cells were incubated for 5 hours with wild type (WT) sFasL or a sFasL mutant (mut sFasL) in which 8 charged amino acids were changed to alanine. Caspase 3/7 activity was measured after incubation with the caspase 3/7 substrate at room temperature for 30 minutes. Data is shown as arbitrary luminescence units. Results show data from three separate experiments, each done in duplicates. Each dot represents individual data. Data analyzed by 2-way ANOVA with Sidak post-hoc analysis. ** p<0.01, *** p<0.001 compared to the respective concentration of mut-sFasL.

FIGS. 2A-D show the mut-sFasL has inhibitory properties. Highly Fas-sensitive Jurkat cells (FIG. 2A), primary human lung small airway epithelial cells (SAEC, FIG. 2B) or murine lung epithelial cells (LA4, FIG. 2D) were incubated with serial dilutions of mutated FasL, (mut sFasL). The 8-site mutated sFasL caused a dose-dependent inhibition of wild type sFasL activity in Jurkat cells (FIG. 2A). The human lung epithelial cells (SAEC) and murine lung epithelial cells (LA4) were less sensitive to WT sFasL (FIG. 2B and FIG. 2D), but an inhibitory effect of the 8-mutant protein was detected. As a confirmatory test, the experiment was repeated using SAEC and an annexin-V translocation assay (FIG. 2C). WT=wild type (no mutant). Results show data from three or four separate experiments, each done in duplicate. Each dot represents individual data; lines represent means±SD. *=P<0.05; **=P<0.01, ***P<0.001 when compared to WT. One way ANOVA with SIdak's post hoc analysis.

FIGS. 3A-C shows the results of a competition assay and binding assay. FIG. 3A shows the results of the competition assay: Serial 3-fold dilutions of either WT or 8-site mutated sFasL were mixed with biotinylated WT sFasL (70 ng/ml). The mixtures were incubated for 2 hours in 96-well plates pre-coated with recombinant hsFas (250 ng/ml). After washing unbound material, biotin was detected using HRP conjugated Streptavidin. FIG. 3B shows the results of the binding assay: Serial 3-fold dilutions of wild type (WT) or mut-sFasL were incubated for 2 hours in wells coated with recombinant human soluble Fas, 5 μg/ml. The sFasL bound to Fas was detected using polyclonal anti-human FasL antibodies. The mut sFasL binding curve was shifted to the left; n=3. FIG. 3C shows the results of Jurkat cells incubated in 96-well plates at a concentration of 1×10⁴ cells/well. Different molar ratios of the Fas-activating antibody CH-11 and mut-sFasL were added, and caspase 3/7 activity measured after 5 hours. The mut-sFasL did not inhibit the activity of CH11. Data represent the results from three separate experiments, each done in duplicate, and were analyzed by 2-way ANOVA with Sidak's post hoc analysis. FIG. 3A shows WT-sFasL compared to mut-sFasL at the same concentrations; FIG. 3B shows comparisons made to no competitor condition for each molecule; and FIG. 3C shows comparisons made to unmixed antibody (Ab). Each dot represents individual data; lines represent means±SD. *=P<0.05; **=P<0.01, ***P<0.001.

FIG. 4 shows the mut-sFasL forms aggregates with the WT sFasL protein. Serial 3-fold dilutions of FLAG tagged mut-sFasL were mixed with biotinylated WT sFasL (60 ng). The mixtures were incubated at room temperature for 15 minutes and applied to streptavidin-coated wells. FLAG tags were detected with HRP anti-FLAG mAb. There was a dose-dependent increase in FLAG signal in wells incubated with the biotinylated WT sFasL+ and the mut-sFasL-FLAG mixture, showing the formation of complexes of WT and mut-sFasL. The mut-sFasL-FLAG without biotin did not bind to the streptavidin-coated plates. Data shown as ratio of the absorbance at 450 nm of each experimental condition to that of media only. Data were generated from three separate experiments, each done in duplicate, and were analyzed by 2-way ANOVA with Dunnet's post hoc analysis; comparisons were made to 0 ng/mL for each condition. Each dot represents individual data, lines represent means±SD. *=P<0.05; **=P<0.01, ***P<0.001.

FIGS. 5A-E show the effects of mut-sFasL on markers of lung inflammation in LPS-treated mice. C57BL/6 mice received oropharyngeal instillations of either PBS or LPS, 2.5 μg/mL. Four hours later some of the mice received oropharyngeal instillations of PBS or mut-sFasL at concentrations of 10 and 100 ng/g and were euthanized 24 hours after the initial LPS/PBS instillations (“early” group). Other mice received the PBS or mut-sFasL (same dose range) 24 hours after LPS/PBS and were euthanized 48 hours after the original LPS instillation (“late” group). N=6 for the groups. In the “late” group, treatment with 100 ng/g mut-sFasL reduced BAL total cells (FIG. 5A), lung homogenate MPO activity (FIG. 5B), and the BAL concentrations of TNF-α, MIP-1β and MCP-1 (FIGS. 5C-E). Data were analyzed by 2-way ANOVA with Dunnet's post hoc analysis; comparisons made to 0 ng/mL for each condition. Data are reported as box plots, in which the box depicts the 25^{th} to 75^{th} percentiles and the lines the min to max range. *=P<0.05; **=P<0.01, ***P<0.001.

FIGS. 6A-D show the effects of mut-sFasL in LPS treated mice. C57BL/6 mice received oropharyngeal instillations of either PBS, or LPS, 2.5 μg/mL. Four hours later some of the mice received oropharyngeal instillations of PBS or mut-sFasL at concentrations of 10 and 100 ng/g and were euthanized 24 hours after the initial LPS/PBS instillations (“early” group). Other mice received the PBS or mut-sFasL (same dose range) 24 hours after LPS/PBS and were euthanized 48 hours after the original LPS instillation (“late” group). N=6 for all groups. Weight loss was similar regardless of treatment (FIG. 6A, percent change from initial weight). The administration of mut-sFasL had no effect total BAL protein (FIG. 6B), BAL IgM (FIG. 6C) or total lung homogenate caspase 3/7 activity (FIG. 6D). Data are reported as box plots, in which the box depicts the 25^{th} to 75^{th} percentiles and the lines the min to max range. Data were analyzed by 2-way ANOVA with Dunnett's post hoc analysis; comparisons were made to 0 ng/mL for each condition.

FIGS. 7A-B show the Fas ligand structure and modifications. FIG. 7A shows the Fas ligand structure. FIG. 7B shows that the Fas ligand is a 281 amino acid protein that contains an intracellular domain, a transmembrane domain (TD), a “stalk region” (SEQ ID NO: 11), and the binding domain. Fas ligand can be cleaved by metalloproteinases at amino acid 103 to form a soluble form that retains activity. Soluble Fas Ligand (sFasL) can be further cleaved at amino acid 128 into a “short” soluble form that is inactive. The charged amino acids of the stalk region were mutated to non-charged alanines (amino acids at positions 4, 7, 8, 11, 13, 14, 20, and 26 of SEQ ID NO: 11).

FIG. 8 shows the predicted protein structures. The top row shows monomeric forms (top) and the bottom row trimeric forms. On the left column, the long (wild type) sFasL monomer shows the stalk region as an alpha helix (blue) which is absent in the short sFasL, lacking the stalk region (right column). Spatial arrangement of the stalk region in the trimeric form of the long sFasL (bottom panel, left) is highlighted by the white ellipses. Mutation of the charged amino acids in the stalk region is predicted to rotate the spatial arrangement of the stalk region (white ellipses) in the trimeric form of mutant sFasL (bottom panel, middle) compared to long sFasL (bottom panel, left). Images of predicted protein structures were generated using Rosetta Molecular Modeling Suite.

FIG. 9 shows representative immunohistochemistry images of lung sections from mice treated with LPS+PBS (top panels) and LPS+mut sFasL (bottom panels) at 48 hr post-LPS instillation. Staining for the neutrophil marker Ly6G (left) and for Caspase-3 (right) are shown. (Bar=100 μm).

FIG. 10 shows the effects of mut-sFasL on caspase 3/7 activity: Effect of increasing concentrations of mut-sFasL on caspase 3/7 activity (white violin bars) or in combination with a fixed concentration of WT sFasL.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if “about 10 and 15” are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease, disorder or condition or at risk for a disease, disorder or condition. The term “patient” includes human and veterinary subjects. In some aspects of the disclosed methods, the “patient” has been diagnosed with a need for treatment, such as, for example, prior to an administering step.

As used herein, the term “comprising” can include the aspects “consisting of” and “consisting essentially of.”

As used herein the terms “amino acid” and “amino acid identity” refers to one of the 20 naturally occurring amino acids or any non-natural analogues that may be in any of the antibodies, variants, or fragments disclosed. Thus “amino acid” as used herein means both naturally occurring and synthetic amino acids. For example, homophenylalanine, citrulline and noreleucine are considered amino acids for the purposes of the invention. “Amino acid” also includes amino acid residues such as proline and hydroxyproline. The side chain may be in either the (R) or the (S) configuration. In some aspects, the amino acids are in the (S) or L-configuration. If non-naturally occurring side chains are used, non-amino acid substituents may be used, for example to prevent or retard in vivo degradation.

“Inhibit,” “inhibiting” and “inhibition” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In an aspect, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels.

“Treatment” and “treating” refer to administration or application of a therapeutic agent (e.g., a decoy peptide or polypeptide described herein) to a subject or performance of a procedure or modality on a subject for the purpose of obtaining a therapeutic benefit of a disease or health-related condition. For example, a treatment may include administration of a pharmaceutically effective amount of a peptide that is capable of inhibiting sFasL activity.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be a pulmonary disease.

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions can be those within the following groups: Ser, Thr, and Cys; Leu, ILe, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, they may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂)ₙCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

As used herein, the term “prevent” or “preventing” refers to preventing in whole or in part, or ameliorating or controlling.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

Based on previous results, it is suggested that the Fas/FasL system plays a role in the pathophysiology of human ARDS and of its animal correlate, acute lung injury (ALI) (Matute-Bello G, et al. J Immunol 163: 2217-2225, 1999; Matute-Bello G, et al. Am J Respir Cell Mol Biol 37: 210-221, 2007; and Perl M, et al. Am J Pathol 167: 1545-1559, 2005). The Fas/FasL system is comprised of the membrane surface receptor Fas (CD95) and its cognate ligand, FasL (CD178). Binding of Fas to FasL activates signaling pathways that lead to apoptosis and also to cytokine release (Farnand A W, et al. Am J Respir Cell Mol Biol 45: 650-658, 2011). FasL is transmembrane homotrimer that can be shed to a soluble form-sFasL-that binds its receptor. It has been found that the main cellular targets of sFasL are alveolar epithelial cells, which respond to Fas ligation with both apoptosis and release of pro-inflammatory cytokines (Bem R A, et al. Am J Physiol Lung Cell Mol Physiol 295: L314-325, 2008; Farnand A W, et al. Am J Respir Cell Mol Biol 45: 650-658, 2011; Matute-Bello G, et al. J Immunol 175: 4069-4075, 2005; and Nakamura M, et al. Am J Pathol 164: 1949-1958, 2004). It has also found that sFasL is present in the bronchoalveolar lavage (BAL) of patients with ARDS, that administration of sFasL to mice and rabbits results in lung injury, and that mice lacking Fas have attenuated alveolar inflammation in response to intratracheal LPS, mechanical ventilation, or viral infection (Matute-Bello G, et al. Am J Physiol Lung Cell Mol Physiol 281: L328-L335, 2001; Matute-Bello G, et al. J Immunol 163: 2217-2225, 1999; Matute-Bello G, et al. Am J Pathol 158: 153-161, 2001; Matute-Bello G, et al. Clin Diagn Lab Immunol 11: 358-361, 2004; van den Berg E, et al. Am J Physiol Lung Cell Mol Physiol 301: L451-L460, 2011). For example, mechanically ventilated mice exposed to LPS develop BAL neutrophilia, which is markedly attenuated in mice deficient in functional Fas (lpr). Furthermore, silencing of Fas attenuates lung injury in mice (Perl M, et al. Am J Pathol 167: 1545-1559, 2005). Thus, the Fas/FasL system is active in the lungs, and plays a role in the development of several different models of acute lung injury.

It was previously shown that the biological activity of sFasL in the lungs is dependent on its structure. Specifically, in the lungs sFasL exists in at least two forms: a 152-amino acid short form, consisting primarily of the binding domain and a short “stalk” region, and a 178 amino-acid long form, consisting of the binding domain plus a longer stalk region (Herrero R, et al. J Clin Invest 121: 1174-1190, 2011). The long form can be cleaved into the short form by proteases such as MMPI (Vargo-Gogola T, et al. Arch Biochem Biophys 408: 155-161, 2002). Interestingly, despite the fact that the short form contains the binding domain, the long form of sFasL is capable of inducing lung injury in mice, which is characterized by increased alveolar permeability, neutrophilic alveolitis, and apoptosis of alveolar epithelial cells (Herrero R, et al. J Clin Invest 121: 1174-1190, 2011). Importantly, the long form of sFasL is the major form present in BAL fluid from patients with ALI/ARDS (Herrero R, et al. J Clin Invest 121: 1174-1190, 2011).

Because both the long and short forms contain the binding domain, it was tested whether the reason for their difference in bioactivity are structural changes resulting from the presence of the stalk region. Therefore, described herein are mutated specific charged amino acid residues in the stalk region of sFasL and the results of how such mutations affect sFasL activity. The results described herein show that the substitution of glutamic acid 116 or glutamic acid 128 by alanine was sufficient to attenuate the activity of sFasL, and substitution of the 8 charged amino acids of the stalk region of sFasL resulted in further loss of function. Additionally, the mutated form of sFasL acted as an inhibitor of the wild type form of sFasL.

### Compositions

Disclosed herein are compositions, including pharmaceutical compositions comprising variants of sFasL. Disclosed herein are compositions, including pharmaceutical compositions capable of inhibiting sFasL activity. Also, disclosed herein are compositions capable of treating a pulmonary disease in a subject.

Disclosed herein are peptides that can comprise or consist of the amino acid sequence of QLFX₁LQX₂X₃LAX₄LX₅X₆STSQMX₇TASSLX₈K (SEQ ID NO: 12), or a variant thereof. In some aspects, X₁ can be H or a non-polar amino acid residue. In some aspects, X₂ can be K or a non-polar amino acid residue. In some aspects, X₃ can be E or a non-polar amino acid residue. In some aspects, X₄ can be E or a non-polar amino acid residue. In some aspects, X₅ can be R or a non-polar amino acid residue. In some aspects, X₆ can be E or a non-polar amino acid residue. In some aspects, X₇ can be H or a non-polar amino acid residue. In some aspects, X₈ can be E or a non-polar amino acid residue. In some aspects, X₂ can be K or a non-polar amino acid residue; X₃ can be E or a non-polar amino acid residue; X₄ can be E or a non-polar amino acid residue; X₅ can be R or a non-polar amino acid residue; X₆ can be E or a non-polar amino acid residue; X₇ can be H or a non-polar amino acid residue; and X₈ can be E or a non-polar amino acid residue. In some aspects, at least one of X₁-X₈ can be a non-polar amino acid residue. In some aspects, at least one of X₁-X₈ can be an alanine residue. In some aspects, each of X₁-X₈ can be a non-polar amino acid residue. In some aspects, each of X₁-X₈ can be an alanine residue.

Disclosed herein are compositions comprising peptides that can comprise the amino acid sequence of SEQ ID NO: 1: QLFHLQKELAELRESTSQMHTASSLEK, wherein one or more of the charged amino acids in SEQ ID NO: 1 can be substituted for a non-polar amino acid residue. In some aspects, the non-polar amino acid residue can be an alanine residue, a glycine residue, a proline residue, a valine residue, a leucine residue, an isoleucine residue, a methionine residue or a tryptophan residue. In some aspects, the non-polar amino acid residue can be an alanine residue, a glycine residue, a proline residue, a valine residue, a leucine residue, an isoleucine residue, a methionine residue, a tryptophan residue or a combination thereof.

Disclosed herein are compositions comprising recombinant, mutant or variant sFAsL polypeptides. In some aspects, one or more of the charged amino acid residues in a stalk region of sFAsL can be substituted for a non-polar amino acid residue. In some aspects, the non-polar amino acid residue can be an alanine residue, a glycine residue, a proline residue, a valine residue, a leucine residue, an isoleucine residue, a methionine residue or a tryptophan residue. In some aspects, the non-polar amino acid residue can be an alanine residue, a glycine residue, a proline residue, a valine residue, a leucine residue, an isoleucine residue, a methionine residue, a tryptophan residue or a combination thereof.

In some aspects, at least two, three, four five, six, seven or eight charged amino acid residues can be replaced by an alanine residue. In some aspects, two, three, four five, six, seven or eight charged amino acid residues in the stalk region of sFAsL can be replaced by an alanine residue.

In any of the compositions described herein the substituted charged amino acid can be a glutamic acid residue, an arginine residue, a histidine residue or a combination thereof.

In some aspects, the peptides can comprise the amino acid sequence QLFALQAALAALAASTSQMATASSLAK (SEQ ID NO: 2). In some aspects, the peptides can comprise the amino acid sequence QLFALQKELAELRESTSQMHTASSLEK (SEQ ID NO: 3). In some aspects, the peptides can comprise the amino acid sequence QLFHLQAELAELRESTSQMHTASSLEK (SEQ ID NO: 4). In some aspects, the peptides can comprise the amino acid sequence QLFHLQKALAELRESTSQMHTASSLEK (SEQ ID NO: 5). In some aspects, the peptides comprise the amino acid sequence QLFHLQKELAALRESTSQMHTASSLEK (SEQ ID NO: 6). In some aspects, the peptides can comprise the amino acid sequence QLFHLQKELAELAESTSQMHTASSLEKK (SEQ ID NO: 7). In some aspects, the peptides can comprise the amino acid sequence QLFHLQKELAELRASTSQMHTASSLEK (SEQ ID NO: 8). In some aspects, the peptides can comprise the amino acid sequence QLFHLQKELAELRESTSQMATASSLEK (SEQ ID NO: 9). In some aspects, the peptides can comprise the amino acid sequence

(SEQ ID NO: 10)
QLFHLQKELAELRESTSQIVIEITASSLAK.

TABLE 1
Amino acid sequences.

| SEQ ID | |
|---|---|
| NO: | Sequence |
| 1 | QLFHLQKELAELRESTSQMHTASSLEK |
| 2 | QLFALQAALAALAASTSQMATASSLAK |
| 3 | QLFALQKELAELRESTSQMHTASSLEK |
| 4 | QLFHLQAELAELRESTSQMHTASSLEK |
| 5 | QLFHLQKALAELRESTSQMHTASSLEK |
| 6 | QLFHLQKELAALRESTSQMHTASSLEK |
| 7 | QLFHLQKELAELAESTSQMHTASSLEKK |
| 8 | QLFHLQKELAELRASTSQMHTASSLEK |
| 9 | QLFHLQKELAELRESTSQMATASSLEK |
| 10 | QLFHLQKELAELRESTSQMHTASSLAK |

In some aspects, the peptides described herein can have at least 80% sequence identity to any of SEQ ID NOs: 1-10. In some aspects, the peptides described herein can have at least 85% sequence identity, at least 90% sequence identify, at least 95% sequence identity, or at least 98% sequence identity to any of SEQ ID NOs: 1-10.

In some aspects, any of the compositions described herein can further comprise a pharmaceutically acceptable carrier. In some aspects, any of the compositions described herein can be formulated for intravenous, subcutaneous or intranasal administration.

Disclosed herein are peptides that can comprise variants of QLFHLQKELAELRESTSQMHTASSLEK (SEQ ID NO: 1). In some aspects, the variants can comprise a sequence having at least 20%, 30%, 40%, 50%, 60%, 70%, 80%, 90%, 95%, 98%, 99% identity to SEQ ID NO: 9. In some aspects, the variants retains at least 50%, 75%, 80%, 85%, 90%, 95% or 99% of the biological activity of the reference protein described herein.

Disclosed herein are peptides that can comprise variants of QLFALQAALAALAASTSQMATASSLAK (SEQ ID NO: 2), QLFALQKELAELRESTSQMHTASSLEK (SEQ ID NO: 3), QLFHLQAELAELRESTSQMHTASSLEK (SEQ ID NO: 4), QLFHLQKALAELRESTSQMHTASSLEK (SEQ ID NO: 5), QLFHLQKELAALRESTSQMHTASSLEK (SEQ ID NO: 6), QLFHLQKELAELAESTSQMHTASSLEKK (SEQ ID NO: 7), QLFHLQKELAELRASTSQMHTASSLEK (SEQ ID NO: 8), QLFHLQKELAELRESTSQMATASSLEK (SEQ ID NO: 9), or QLFHLQKELAELRESTSQMHTASSLAK (SEQ ID NO: 10). In some aspects, the variants can comprise a sequence having at least 20%, 30%, 40%, 50%, 60%, 70%, 80%, 90%, 95%, 98%, 99% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 10, or SEQ ID NO: 12. In some aspects, the variants retains at least 50%, 75%, 80%, 85%, 90%, 95% or 99% of the biological activity of the reference protein described herein.

As used herein, the term “peptide” refers to a linear molecule formed by binding amino acid residues to each other via peptide bonds. As used herein, the term “polypeptide” refers to a polymer of (the same or different) amino acids bound to each other via peptide bonds.

In some aspects, the decoy peptide or polypeptide can be of any length so long as the peptides described herein can inhibit sFasL activity.

In some aspects, the peptides described herein can further comprise 1, 2, 2, 4, 5, 10, 15, 20, 25, 30, 35, 40, 45, 50 amino acid residues at the N-terminal end of the disclosed peptides. In some aspects, the peptides described herein can further comprise 1, 2, 2, 4, 5, 10, 15, 20, 25, 30, 35, 40, 45, 50 amino acid residues at the C-terminal end of the disclosed peptides disclosed herein. In some aspects, the amino acid residues that can be present at either the N-terminal end or the C-terminal end of any of the peptides disclosed herein can be unimportant for inhibiting the inhibition of sFasL activity. In some aspects, the amino acid residues added to the N-terminal end or the C-terminal end of the peptides disclosed herein may prevent ubiquitination, improve stability, help maintain the three dimensional structure of the peptide, or a combination thereof.

In some aspects, the peptides disclosed herein can further comprise a peptide having one or more amino acid residues with a modified side chain. In some aspects, one or more amino acids of any of the peptides disclosed here can have a modified side chain. Examples of side chain modifications include but are not limited to modifications of amino acid groups, such as reductive alkylation; amidination with methylacetimidate; acylation with acetic anhydride; carbamolyation of amino groups with cynate; trinitrobenzylation of amino acid with 2,4,6-trinitrobenzene sulfonic acid (TNBS); alkylation of amino groups with succinic anhydride; and pyridoxylation with pridoxal-5-phosphate followed by reduction with NaBH₄.

In some aspects, the guanidine group of the arginine residue may be modified by the formation of a heterocyclic condensate using a reagent, such as 2,3-butanedione, phenylglyoxal, and glyoxal. In some aspects, the carboxyl group may be modified by carbodiimide activation via O-acylisourea formation, followed by subsequent derivatization, for example, to a corresponding amide.

In some aspects, the sulfhydryl group may be modified by methods, such as carboxymethylation with iodoacetic acid or iodoacetamide; performic acid oxidation with cysteic acid; formation of mixed disulfides by other thiol compounds; a reaction by maleimide, maleic anhydride, or other substituted maleimide; formation of mercury derivatives using 4-chloromercuribenzoate, 4-chloromercuriphenylsulfonic acid, phenylmercury chloride, 2-chloromercuri-4-nitrophenol, and other mercurial agents; and carbamolyation with cyanate at alkaline pH. In addition, the sulfhydryl group of cysteine may be substituted with a selenium equivalent, whereby a diselenium bond may be formed instead of at least one disulfide bonding site in the peptide.

In some aspects, the tryptophan residue may be modified by, for example, oxidation with N-bromosuccinimide or alkylation of the indole ring by 2-hydroxy-5-nitrobenzyl bromide or sulfonyl halide. Meanwhile, the tyrosine residue may be modified by nitration using tetranitromethane to form a 3-nitrotyrosine derivative.

In some aspects, the modification of the imidazole ring of the histidine residue may be accomplished by alkylation with an iodoacetic acid derivative or N-carbethoxylation with diethylpyrocarbonate.

In some aspects, the proline residue may be modified by, for example, hydroxylation at the 4-position.

In some aspects, the peptides described herein can be further modified to improve stability. In some aspects, any of the amino acid residues of the peptides described herein can be modified to improve stability. In some aspects, peptide can have at least one amino acid residue that has an acetyl group, a fluorenylmethoxy carbonyl group, a formyl group, a palmitoyl group, a myristyl group, a stearyl group, or polyethylene glycol. In some aspects, an acetyl protective group can be bound to the peptide described herein.

As used herein, the term “stability” refers to storage stability (e.g., room-temperature stability) as well as in vivo stability. The foregoing protective group can protect the peptides described herein from the attack of protein cleavage enzymes in vivo.

As used herein, the term “peptide” can also be used to include functional equivalents of the peptides described herein. As used herein, the term “functional equivalents” can refer to amino acid sequence variants having an amino acid substitution, addition, or deletion in some of the amino acid sequence of the decoy peptide or polypeptide while simultaneously having similar or improved biological activity, compared with the peptide as described herein. In some aspects, the amino acid substitution can be a conservative substitution. Examples of the naturally occurring amino acid conservative substitution include, for example, aliphatic amino acids (Gly, Ala, and Pro), hydrophobic amino acids (Ile, Leu, and Val), aromatic amino acids (Phe, Tyr, and Trp), acidic amino acids (Asp and Glu), basic amino acids (His, Lys, Arg, Gln, and Asn), and sulfur-containing amino acids (Cys and Met). In some aspects, the amino acid deletion can be located in a region that is not directly involved in the activity of the decoy peptide and polypeptide disclosed herein.

In some aspects, the amino acid sequence of the peptides described herein can include a peptide sequence that has substantial identity to any of sequence of the peptides disclosed herein. As used herein, the term “substantial identity” means that two amino acid sequences, when optimally aligned and then analyzed by an algorithm normally used in the art, such as BLAST, GAP, or BESTFIT, or by visual inspection, share at least about 60%, 70%, 80%, 85%, 90%, or 95% sequence identity. Methods of alignment for sequence comparison are known in the art.

In some aspects, the amino acid sequence of the peptides described herein can include a peptide sequence that has some degree of identity or homology to any of sequences of the peptides disclosed herein. The degree of identity can vary and be determined by methods known to one of ordinary skill in the art. The terms “homology” and “identity” each refer to sequence similarity between two polypeptide sequences. Homology and identity can each be determined by comparing a position in each sequence which can be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. The peptides described herein can have at least or about 25%, 50%, 65%, 75%, 80%, 85%, 90%, 95%, 96%, 97%, 98%, or 99% identity or homology to the decoy peptide or polypeptide, wherein the peptide is one or more of SEQ ID NOs: 1-10.

### Pharmaceutical Compositions

As disclosed herein, are pharmaceutical compositions, comprising the peptides described herein. Also disclosed herein, are pharmaceutical compositions, comprising the peptides described herein and a pharmaceutical acceptable carrier. Further disclosed herein are pharmaceutical compositions for treating a pulmonary disease; or inhibiting sFasL activity in a subject. In some aspects, the pharmaceutical compositions can comprise: a) a therapeutically effective amount of the peptides described herein; and b) a pharmaceutically acceptable carrier.

The pharmaceutical compositions described above can be formulated to include a therapeutically effective amount of a peptide disclosed herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a pulmonary disease.

The pharmaceutical compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with or diagnosed with a pulmonary disease in an amount sufficient to at least partially improve a sign or symptom or to inhibit the progression of (and preferably arrest) the symptoms of the condition, its complications, and consequences (e.g., developing a pulmonary disease). An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effect amount includes amounts that provide a treatment in which the onset or progression of a pulmonary disease or a symptom of a pulmonary disease is ameliorated. One or more of the symptoms can be less severe. Recovery can be accelerated in an individual who has been treated.

In some aspects, the pharmaceutical composition can be formulated for intravenous administration. In some aspects, the pharmaceutical composition can be formulated for subcutaneous, intranasal, oropharyngeal or oral administration. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the peptides disclosed herein. Thus, compositions can be prepared for parenteral administration that include the peptides dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

### Methods of Treatment

Disclosed herein are methods of treating a pulmonary disease in a subject. In some aspects, the methods can comprise: a) administering to a subject a therapeutically effective amount of any of the peptides or polypeptides disclosed herein. In some aspects, the methods can further comprise: b) a pharmaceutically acceptable carrier.

Disclosed herein are methods of inhibiting sFasL activity in a subject. In some aspects, the methods can comprise: a) administering to a subject a therapeutically effective amount of any of the peptides disclosed herein. In some aspects, the methods can comprise: a) administering to a subject a therapeutically effective amount of a compositions comprising any of the peptides disclosed herein and a pharmaceutically acceptable carrier.

Disclosed herein are methods of ameliorating one or more symptoms of a pulmonary disease. In some aspects, the methods can comprise: administering to a subject a therapeutically effective amount of any of the peptides disclosed herein. In some aspects, the methods can comprise administering to a subject a therapeutically effective amount of any of the peptides disclosed herein in or with a pharmaceutically acceptable carrier. In some aspects, the one or more symptoms of a pulmonary disease can be shortness of breath, cough, wheezing or a combination thereof. ARDS can cause acute respiratory failure with hypoxemia and respiratory acidosis that can require mechanical ventilation. In some aspects, the one or more symptoms of a pulmonary disease can be acute respiratory failure with hypoxemia, respiratory acidosis or a combination thereof.

In any of the methods disclosed herein, the pulmonary disease can be obstructive pulmonary disease (COPD), emphysema, asthma, idiopathic pulmonary fibrosis, pneumonia, tuberculosis, cystic fibrosis, bronchitis, pulmonary hypertension, interstitial lung disease, acute respiratory distress syndrome (ARDS), acute lung injury (ALI), idiopathic pulmonary pneumonitides, or lung cancer. In some aspects, the pulmonary disease can be acute respiratory distress syndrome. In some aspects, the pulmonary disease can be acute lung injury.

In some aspects, the methods can comprise administering a composition that can be formulated for intravenous, subcutaneous, intranasal, oropharyngeal or oral administration.

In some aspects, the subject can be identified as being in need of treatment before the administration step. In some aspects, the subject can have a pulmonary disease.

Amounts effective for this use can depend on the severity of the condition, disease or disease or the severity of the risk of the condition, disease or disorder, and the weight and general state and health of the subject, but generally range from about 0.05 μg to about 1000 μg (e.g., 0.5-100 μg) of an equivalent amount of the peptide per dose per subject. Suitable regimes for initial administration and booster administrations are typified by an initial administration followed by repeated doses at one or more hourly, daily, weekly, or monthly intervals by a subsequent administration. For example, a subject can receive peptides in the range of about 0.05 to 1,000 μg equivalent dose per dose one or more times per week (e.g., 2, 3, 4, 5, 6, or 7 or more times per week). For example, a subject can receive 0.1 to 2,500 μg (e.g., 2,000, 1,500, 1,000, 500, 100, 10, 1, 0.5, or 0.1 μg) dose per week. A subject can also receive peptides in the range of 0.1 to 3,000 μg per dose once every two or three weeks. A subject can also receive 2 mg/kg every week (with the weight calculated based on the weight of the peptide.

The total effective amount of the peptides disclosed herein in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month). Alternatively, continuous intravenous infusions sufficient to maintain therapeutically effective concentrations in the blood are also within the scope of the present disclosure.

The therapeutically effective amount of the peptides present within the compositions described herein and used in the methods as disclosed herein applied to mammals (e.g., humans) can be determined by one of ordinary skill in the art with consideration of individual differences in age, weight, and other general conditions (as mentioned herein).

Kits

The kits can comprise one or more of the peptides or pharmaceutical compositions disclosed herein. The peptides or compositions described herein can be packaged in a suitable container labeled, for example, for use to treat a pulmonary disease including but not limited to obstructive pulmonary disease (COPD), emphysema, asthma, idiopathic pulmonary fibrosis, pneumonia, tuberculosis, cystic fibrosis, bronchitis, pulmonary hypertension, interstitial lung disease, acute respiratory distress syndrome (ARDS), acute lung injury (ALI), idiopathic pulmonary pneumonitides or lung cancer. Accordingly, packaged products (e.g., sterile containers containing the composition described herein and packaged for storage, shipment, or sale at concentrated or ready-to-use concentrations) and kits, including at least one or more of the peptides as described herein and instructions for use, are also within the scope of the disclosure. A product can include a container (e.g., a vial, jar, bottle, bag, or the like) containing the peptides or compositions described herein. In addition, the kits further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The peptides or compositions can be ready for administration (e.g., present in dose-appropriate units), and may include a pharmaceutically acceptable adjuvant, carrier or other diluent. Alternatively, the compounds can be provided in a concentrated form with a diluent and instructions for dilution.

### EXAMPLES

### Example 1: The Bioactivity of Soluble Fas Ligand is Modulated by Amino Acids of its Stalk Region

Abstract. It has been reported that the 26-amino acid N-terminus stalk region of soluble Fas ligand (sFasL), which is separate from its binding site, is required for its biological function. Described herein are the results of an investigation of the mechanisms that link the structure of the stalk region with sFasL function. Mutation of the 8 charged amino acids of the stalk region into the non-charged amino acid alanine (mut-sFasL) resulted in reduced apoptotic activity compared to wild type sFasL (WT-sFasL). Furthermore, the mut-sFasL attenuated WT-sFasL function on the Fas-sensitive human T-cell line Jurak and on primary human small airway epithelial cells (SAEC). The mut-sFasL attenuated WT-sFasL function on the Fas-sensitive human T-cell line Jurak and on primary human small airway epithelial cells. The inhibitory mechanism was associated with the formation of complexes of the mut-sFasL with the WT protein. Furthermore, intratracheal administration of the mut-sFasL to mice 24 hours after intratracheal Escherichia coli lipopolysaccharide (LPS) resulted, 24 hours later, in attenuation of the inflammatory response. It was concluded that changes in the structure of the stalk region of sFasL result in mutant variants that interfere with the WT protein function in vitro and in vivo.

Introduction. The acute respiratory distress syndrome (ARDS) is defined by the sudden onset of bilateral lung infiltrates and impaired gas exchange, in the absence of evidence of left ventricular dysfunction (Bernard G A, et al. Consensus Committee. J Crit Care. 1994; 9(1):72-81; and Ranieri V M, et al. JAMA. 2012; 307(23):2526-33). ARDS is a clinical problem in the United States, affecting 200,000 patients per year and resulting in death of approximately 75,000 persons (Rubenfeld G D, et al. N Engl J Med. 2005; 353(16):1685-93). Over the past 20 years, the mortality due to ARDS has decreased from approximately 60% to 30-40%, in part because of the discovery that mechanical ventilation with lower tidal volumes is protective; however, mortality remains unacceptably high (Li G, et al. Am J Respir Crit Care Med. 2011; 183(1):59-66). Furthermore, there is increasing evidence that survivors suffer significant long-term consequences (Herridge M S, et al. Intensive Care Med. 2016; 42(5):725-38). Despite these negative outcomes, specific treatments are lacking.

Data suggest that the Fas/FasL system plays a role in the pathophysiology of human ARDS and of its animal correlate, acute lung injury (ALI) (Matute-Bello G, et al. J Immunol. 1999; 163:2217-25; Matute-Bello G, et al. Am J Respir Cell Mol Biol. 2007; 37(2):210-21; Perl M, et al. Am J Pathol. 2005; 167(6):1545-59; Matute-Bello G, et al. Am J Pathol. 2001; 158(1):153-61; Matute-Bello G, et al. Clin Diagn Lab Immunol. 2004; 11(2):358-61. PubMed PMID: 15013988; and Matute-Bello G, et al. Am J Physiol Lung Cell Mol Physiol. 2001; 281:L328-L35). The Fas/FasL system is comprised of the membrane surface receptor Fas (CD95) and its cognate ligand, FasL (CD178). In the lungs, Fas is expressed in the airway and alveolar epithelia, in fibroblasts and in alveolar macrophages (Nakamura M, et al. Am J Pathol. 2004; 164(6):1949-58). Binding of FasL to Fas activates signaling pathways that lead to apoptosis and also to cytokine release (Farnand A W, et al. Am J Respir Cell Mol Biol. 2011; 45(3):650-8). For example, in alveolar epithelial cells, Fas activation can lead to apoptosis, but also to cytokine release via adapter proteins such as MyD88. FasL is expressed on cell membranes as a transmembrane protein that can be cleaved to a soluble form-sFasL-that binds its receptor. FasL, soluble or membrane-bound, must form homotrimers in order to activate Fas (Holler N, et al. Mol Cell Biol. 2003; 23(4):1428-40). In the lungs, the main cellular targets of sFasL are alveolar epithelial cells, which respond to Fas ligation with both apoptosis and release of pro-inflammatory cytokines (Nakamura M, et al. Am J Pathol. 2004; 164(6):1949-58; Farnand A W, et al. Am J Respir Cell Mol Biol. 2011; 45(3):650-8; Matute-Bello G, et al. J Immunol. 2005; 175(6):4069-75; and Bem R A, et al. Am J Physiol Lung Cell Mol Physiol. 2008; 295(2):L314-25). Also, sFasL is present in the bronchoalveolar lavage (BAL) fluid of patients with ARDS, and administration of sFasL to mice and rabbits results in lung injury, and that mice lacking Fas have attenuated alveolar inflammation in response to intratracheal lipopolysaccharide (LPS), mechanical ventilation, or viral infection (Matute-Bello G, et al. J Immunol. 1999; 163:2217-25; Matute-Bello G, et al. Am J Pathol. 2001; 158(1):153-61; Matute-Bello G, et al. Clin Diagn Lab Immunol. 2004; 11(2):358-61. PubMed PMID: 15013988; Matute-Bello G, et al. Am J Physiol Lung Cell Mol Physiol. 2001; 281:L328-L35; and van den Berg E, et al. Am J Physiol Lung Cell Mol Physiol. 2011; 301(4):L451-L60). For example, mechanically ventilated mice exposed to LPS develop BAL neutrophilia, which is markedly attenuated in mice deficient in functional Fas (B6.MRL-Fas^{lpr}/J, “lpr” mice) (Gil S, Farnand A W, et al. Respir Res. 2012; 13:91). Furthermore, silencing of Fas attenuates lung injury in mice (Perl M, et al. Am J Pathol. 2005; 167(6):1545-59). Thus, the Fas/FasL system is active in the lungs and plays a role in the development of several different models of acute lung injury. In addition to the lungs, in humans mutations in either Fas or FasL are associated with a syndrome of immune dysregulation characterized by cytopenias, predisposition to infections and lymphoproliferative disorders known as “autoimmune lymphoproliferative disorder”, or ALPS (Rieux-Laucat F, et al. J Clin Immunol. 2018; 38(5):558-68).

the biological activity of sFasL in the lungs is dependent on its structure (Herrero R, et al. J Clin Invest. 2011; 121(3):1174-90). Specifically, in the lungs, sFasL exists in at least two forms: a 152-amino acid short form, consisting primarily of the Fas binding domain and a short “stalk” region, and a 178 amino-acid long form, consisting of the Fas binding domain plus a longer stalk region (Herrero R, et al. J Clin Invest. 2011; 121(3):1174-90). The long form can be cleaved into the short form by proteases such as MMP7 (Vargo-Gogola T, et al. Arch Biochem Biophys. 2002; 408(2):155-61). Interestingly, despite the fact that the short form contains the binding domain, it is the long form of sFasL that is capable of inducing lung injury in mice, which is characterized by increased alveolar permeability, neutrophilic alveolitis, and apoptosis of alveolar epithelial cells (Herrero R, et al. J Clin Invest. 2011; 121(3):1174-90). Importantly, the long form of sFasL is the major form present in BAL fluid from patients with ARDS (Herrero R, et al. J Clin Invest. 2011; 121(3):1174-90).

Because both the long and short forms of sFasL contain the Fas binding domain, it was tested whether the difference in bioactivity is a structural change resulting from the presence or absence of the stalk region. Therefore, specific charged amino acid residues were mutated in the stalk region and it was determined whether such mutations affect sFasL activity. The results show that the substitution of the 8 charged amino acids of the stalk region resulted in significant loss of function, confirming the important role of the stalk region in the bioactivity of sFasL. The responses seen in LPS-treated mice exposed to the mutated form of sFasL (mut-sFasL) were similar to the published phenotype of ventilated mice lacking functional Fas (lpr) following LPS exposure (Gil S, et al. Respir Res. 2012; 13:91).

Methods. Cells and cell culture. To test Fas responses in vitro, the highly-Fas sensitive cell line, Jurak, was used. Jurak cells are human T lymphocyte cells first isolated from the peripheral blood of a 14 year old boy with acute cell leukemia (Gillis S and Watson J. J Exp Med. 1980; 152(6):1709-19). Jurak cells are exquisitely sensitive to Fas ligation and are commonly used as a model cell in studies involving the Fas/FasL system; here they are used as such, rather than as a model of T-cell responses. Jurak cells (human leukemic T lymphocytes) were purchased from ATCC (Clone TIB-152) and grown at 37° C. and 5% CO₂ in RPMI-1640 medium supplemented with 10% heat-inactivated fetal bovine serum. Cells were authenticated using short tandem repeat analysis. Mycoplasma status was weakly positive by PCR (Venor GeM Mycoplasma Detection Kit, Sigma-Aldrich, Cat #MP0025). The Jurak data was confirmed in primary human lung epithelial cells and the murine alveolar epithelial cell line LA4, which are described herein.

Small airway epithelial cells (SAEC) are primary human cells isolated from the 1-mm bronchiole area of normal human lung tissue, and were purchased from Lonza (Walkersville, Md., catalogue number CC-2547, lot number 0000669507). Cells used came from a single donor and confirmed mycoplasma-free by the vendor. Cells were grown at 37° C., 5% CO₂ in small airway epithelial basal medium (SAGM, Lonza, Cat #CC-3119 CC-3119) supplemented with SAGM SingleQuots (Lonza, catalogue number CC-4124), and used between passages 2 and 4.

Mouse lung epithelial LA-4 cells were obtained from ATCC (catalogue number CCL-196, lot number 3404534). This cell line is derived from a A/He mouse lung adenoma (Stoner G, et al. Cancer Res. 1975; 35(8):2177-85). The lot was certified as mycoplasma-free by the vendor. The cells were grown at 37° C., 5% CO₂ in F-12K medium supplemented with 15% heat-inactivated fetal bovine serum.

FreeStyle 293-F cells are human embryonic kidney cells used as a mammalian expression system, and were obtained from Invitrogen (catalogue number P/N 51-0029, lot number 1038913). The cells were grown in FreeStyle 293 expression medium at 37° C. and 8% CO₂ while shaking at 135 rpm. Mycoplasma status was negative as determined by PCR.

Antibodies and Reagents. To prepare immunoaffinity columns, mouse monoclonal anti-human FasL antibodies, clone 100419 (R&D System, Cat #MAB126, RRID:AB 2246667, Minneapolis, Minn.), were used. For the Fas-FasL binding assay, recombinant human soluble Fas receptors (PeproTech, Rocky Hill, N.J.) and biotinylated goat anti-human FasL antibodies (PeproTech Cat #500-P184bt-50 ug, RRID:AB 148184) were used. Mouse anti-human Fas (CD95) monoclonal IgM antibody, clone CH-11 and its isotype control antibody, clone 3D12, were obtained from MBL, Nagoya, Japan (MBL International Cat #SY-001, RRID:AB 591016 and Cat #M077-3, RRID:AB 593057, respectively). FLAG tagged mut-sFasL was detected using HRP anti-FLAG monoclonal antibody (Sigma-Aldrich, Cat #A85920). Unless otherwise indicated, the other chemicals were purchased from Sigma-Aldrich (St. Louis, Mo.).

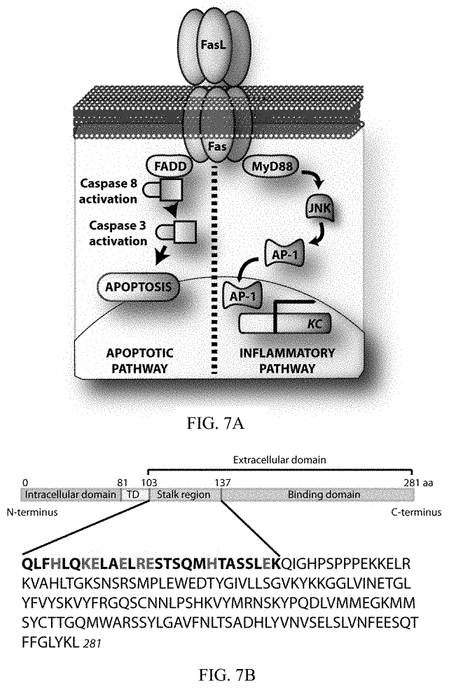

Cloning of human sFasL cDNA and site-directed mutagenesis. Human sFasL cDNA sequences coding amino acids 103 to 281 were amplified with DNA polymerase (Easy-A High-Fidelity PCR Cloning Enzyme, Agilent Technologies, Cat #600400, La Jolla, Calif.) using cloned human sFasL cDNA (ATCC 10659567) as a template and cloned into pSecTag/FRT/V5-His-TOPO mammalian expression vector (Invitrogen, Cat #K6025-01, Carlsbad, Calif.) (FIG. 7). Although this vector had a V5-His tag coding region on the 3′-end of the cloning site, the expressed sFasL should have been the native forms because the amplified sFasL cDNA had its own native stop codons. Site-directed mutagenesis in the plasmid encoding sFasL cDNA were performed using a QuikChange II site-directed mutagenesis kit (Agilent Technologies, Cat #200523-5). FLAG-tag coding cDNA was cloned into 3′-end of 281th amino acid coding sequence of 8-mut sFasL cDNA sequence in frame to produce 8-mut sFasL-FLAG expression plasmid (pSec mut sFasL-FLEG). The sequences of the cloned pSecTag/cDNA-sFasL plasmids were confirmed by commercial DNA sequencing services by Eurofins MWG/Operon (Huntsville, Ala.).

Expression of sFasL protein in mammalian cells. Suspensions of human embryonic kidney cells (FreeStyle 293-F cells, Invitrogen) were transfected with pSecTag/cDNA-sFasL encoding the wild type human sFasL cDNA and the 8-site mutated human sFasL cDNA using 293fectin lipofection reagent (Invitrogen) and incubated in a humidified incubator at 8% CO₂, 37° C. with the FreeStyle293 Expression serum-free medium (Gibco, Carlsbad, Calif., Cat #12338-018) while shaking. After 6 days of incubation, cell supernatants were harvested, filtered with a 0.22 μm nitrocellulose membrane and stored at −20° C. until used. For sFasL protein purification, immunoaffinity columns (Hi Trap NETS-activated HP, GE Healthcare, Piscataway, N.J., Cat #17071601) were used, in which mouse monoclonal antibodies to human FasL (R&D System, Clone: 100419, Minneapolis, Minn.) were immobilized on agarose beads. The concentrations of purified human sFasL proteins were measured by a human sFasL ELISA kit (MBL, Nagoya, Japan, Cat #5255) following the manufacturer's instructions.

Biotinylation of sFasL. Immunoaffinity purified WT-sFasL was biotinylated with No-Weight Format EZ-Link Sulfo-NHS-LC-Biotin (Pierce, Rockford, Ill., Cat #21327) following the manufacturer's instructions. To capture biotinylated WT-sFasL in the mixtures of biotinylated WT-sFasL and mut-sFasL-FLAG, Streptavidin-variant (Strep-Tactin XT) coated 96-well plates (iba, Gottingen, Germany, Cat #2-4101-001) were used.

Measurements of apoptotic cascade activation. Apoptotic cascade activation was assessed by determining caspase 3/7 activity and Annexin V externalization. Caspase-3/7 activity was measured using a commercial substrate (Caspase-Glo 3/7 Assay, Promega, Madison, Wis., Cat #G8090) following manufacturer's instructions. Annexin V was measured with a luminescent commercial assay that allows for measurement of annexin V externalization in adherent cells (RealTime-Glo Annexin V assay, Promega, Madison, Wis., Cat #JA1011). Luminescence was measured with a Synergy H4 Microplate Reader (BioTek, Winooski, Vt.).

Animal studies. Mice were housed in conventional cages with ad libitum access to tap water and standard chow, in humidity-controlled (50%) and temperature-controlled (22° C.-23° C.) rooms with 12 hours/12 hours light/dark cycles. In the morning, male C57BL/6J mice (Jackson Laboratories, Bar Harbor Me.) weighing 24-30 were anesthetized with 4% inhaled isoflurane and randomized to treatment for one oropharyngeal instillations of either PBS, or LPS, 2.5 μg/mL (Sigma-Aldrich, St. Louis Mo.) (Time=0 hr). The PBS group was instilled first to avoid cross-contamination with LPS. The mice were allowed to recover from anesthesia, given full access to food and water. The mice were allowed to recover from anesthesia and given full access to food and water. Some of the mice received oropharyngeal instillations of mut-sFasL at 4 hr and were euthanized at 24 hr (“early” group). Other mice received oropharyngeal mut-sFasL at 24 hours and were euthanized at 48 hr (“late” group). In both cases, mut-sFasL doses were 0, 30 and 100 ng/mL. The number of mice used per group was 6, and the total number of mice was 54.

Following instillations, the mice were monitored for signs of discomfort every 15 minutes during the first hour, and then every four hours during daytime. The primary monitoring parameters were loss of prone posture, loss of movement with mild physical stimulation, weight loss of >20% (weight was measured once per day) and severe respiratory distress. Secondary monitoring parameters included ruffled fur, nasal or ocular discharge and an abnormal respiratory pattern. The presence of one major parameter or two minor parameters was considered an indication for euthanasia, but no mice met these criteria.

At the end of the experiments, the mice were euthanized with intraperitoneal injections of euthanasia-D (1.56 mg/g of pentobarbital component) and exsanguinated by severing the inferior vena cava. The thorax was opened rapidly, the trachea was cannulated with a 20-gauge catheter, the left hilum was clamped and the left lung removed and flash-frozen in liquid nitrogen for homogenization, and the right lung was subjected to bronchoalveolar lavage (BAL) (van den Berg E, et al. Am J Physiol Lung Cell Mol Physiol 301: L451-L460, 2011). In some animals, the right lung was also fixed in 4% paraformaldehyde at an inflation pressure of 15 cm H₂O and embedded in paraffin for subsequent histological studies.

BAL and lung homogenate measurements: Total cell counts in the BAL fluid were performed with an automated cell counter (Cellometer, Nexcelom, Lawrence, Mass.). Differential counts were performed on cytospin preparations using the Diff-quick method (Fisher Scientific Company L.L.C., Kalamazoo, Mich.). Total protein was measured with the bicinchoninic acid method (BCA assay, Pierce, Rockford, Ill.). Tissue polymorphonuclear cells (PMNs) were assessed by myeloperoxidase (MPO) activity measured in lung homogenates using the Amplex red peroxidase assay kit (Invitrogen, Carlsbad, Calif.). BAL fluid cytokines were measured using a fluorescent-bead based luminex multiplex reader (Bioplex 200, Biorad, Hercules, Calif.) and multiplex cytokine beads (Invitrogen, Carlsbad, Calif.).

Statistical Analysis. Quantitative variables were expressed as mean±SD. Analysis of multiple groups was done with one-way ANOVA when one factor was tested, or 2-way ANOVA when two factors were tested. Significance between groups was determined by Sidak's post-hoc analysis for one-way ANOVA or Dunnett's analysis for 2-way ANOVA. A p value less than 0.05 was considered statistically significant. The statistical analyses were performed using GraphPad Prism software version 9.0.0.

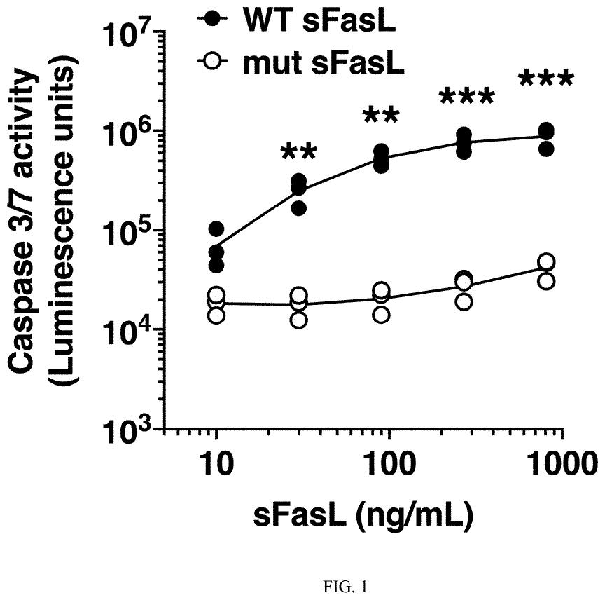

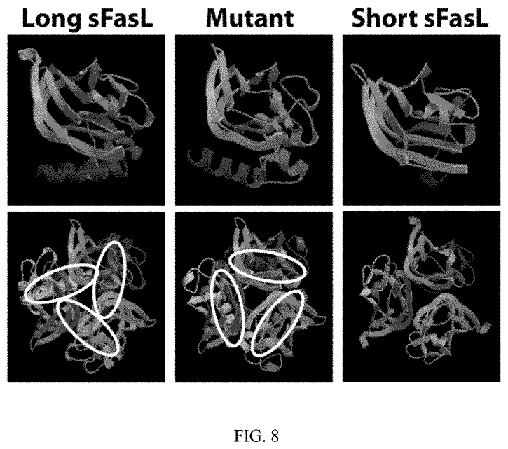

Results. Mutations of the charged amino acids in the stalk region of sFasL influence its biological activity in vitro. To test whether the structure of the stalk region of sFasL influences its biological activity, a mutant sFasL molecule (mut-sFasL) was cloned in which 8 charged amino acids in the 26 amino acid stalk region were changed to the non-charged amino acid alanine (FIG. 7). DNA sequencing data for the FasL constructs was also carried out: DNA sequence of wild type sFasL construct and eight different clones. The mutant sFasL clone used in the study is clone 1. The 8 clones results in mutations at H106A, K109A, E110A, E113A, R115A, El 16E, H122A and E128A of the Fas ligand or H4A, K7A, E8A, El 1A, R13A, E14E, H20A and E26A of the stalk region (SEQ ID NO: 11). Using the Rosetta Molecular Modeling Suite at the Institute for Protein Design (https://rosbetta.bakerlab.org/), the structure of the mut sFasL monomer and trimer were modeled, illustrating the predicted conformational changes in the stalk region when the charged residues are changed to alanine (FIG. 8). Then, it's the peptides ability to activate caspases 3/7, an important step in the apoptosis cascade, was tested. Human Jurkat t-cells were used because they are highly sensitive to Fas activation and therefore are a good tool to test the Fas/FasL system. It was found that the mut-sFasL had significantly reduced caspase 3/7 activity compared to WT-sFasL (FIG. 1). The mutant-sFasL retained some bioactivity at the highest concentration tested (810 ng/mL) but its activity was approximately two orders of magnitude less than the wild-type sFasL (p=0.04 for mutant sFasL at 910 ng/mL vs 10 ng/mL).

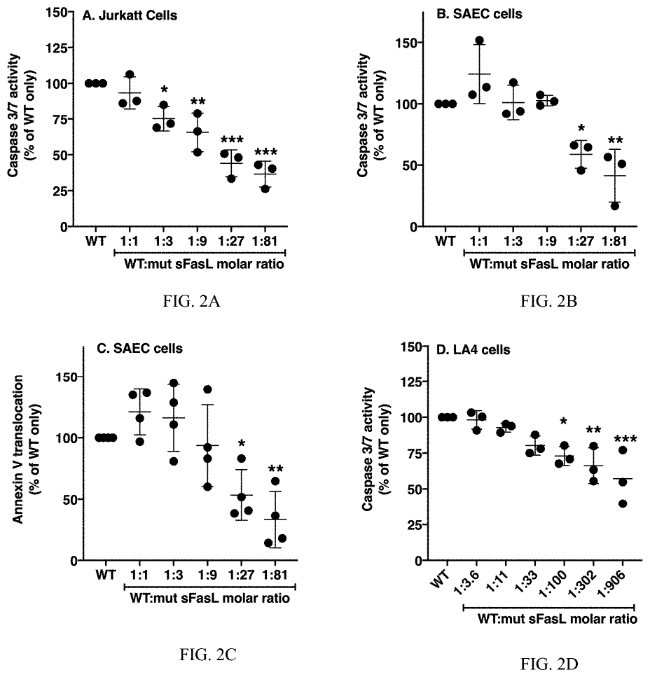

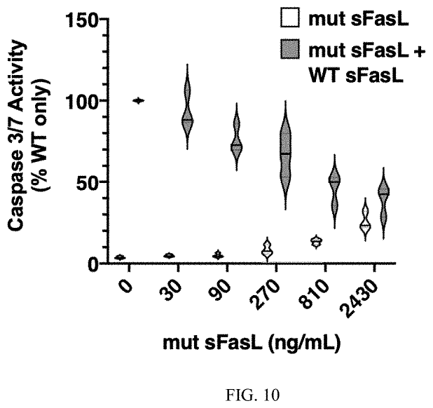

The mut-sFasL attenuates the activity of WT-sFasL. Then, the caspase 3/7 activity of Jurkat cells incubated with different molar ratios of WT and mut-sFasL was investigated. The results show that the mut-sFasL significantly attenuated WT-sFasL activity at molar ratios greater than 1:3 (FIG. 2A; FIG. 10). The effect of increasing concentrations of mut-sFasL on caspase 3/7 activity or in combination with a fixed concentration of WT sFasL was observed at the highest dose of 2430 ng/mL, corresponding to the 1:81 WT:mut sFasL molar ratio in FIG. 2, the mut-sFasL leads to intrinsic caspase-3/7 activation. The molar mixtures on primary human small airway epithelial cells (SAEC) were tested. These cells are commercially isolated from the distal airways of human lungs. Again, an inhibitory effect of the mut-sFasL on the ability of WT-sFasL to induce caspase 3-7 activity was observed, although at higher molar ratios (FIG. 2B). The experiment using SAEC and an alternative measurement of apoptotic activation, annexin-V externalization on the cell membrane, was then repeated. Again, an attenuation of WT-sFasL activity at molar ratios of 1:27 or higher (FIG. 2C) was demonstrated. Finally, it was found that mut-sFasL also inhibited the ability of WT-sFasL to induce caspase 3-7 activity in murine lung epithelial cells, although these cells are less sensitive to human FasL (FIG. 2D).

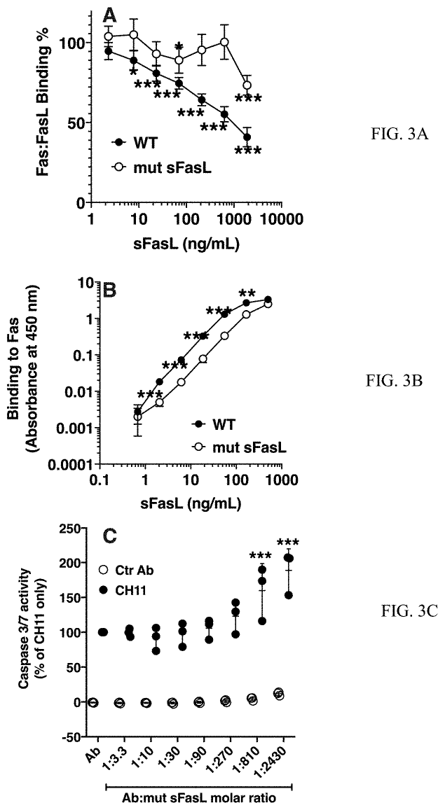

WT-sFasL inhibition occurs prior to receptor binding. It was then investigated whether the mut-sFasL attenuated WT-sFasL activity by competing for the Fas receptor. Recombinant human Fas at 250 ng/mL were coated and the binding of a fixed concentration of biotinylated WT-sFasL (70 ng/mL) were measured in the presence of serial 3-fold dilutions of either WT-sFasL or mut-sFasL. As the concentration of non-biotinylated WT-sFasL increased, binding of biotinylated WT-sFasL to Fas decreased, indicating competition for the Fas receptor (FIG. 3A). However, increasing concentrations of mut-sFasL had little effect on binding of biotinylated WT-sFasL, except at very high doses. This finding was surprising and suggested that the mut-sFasL attenuates WT-sFasL activity by a mechanism other than competition for the receptor. It was then investigated whether the two sFasL molecules differ in their ability to bind directly to immobilized Fas. Serial dilutions of either WT-sFasL or mut-sFasL were incubated in wells coated with recombinant human Fas, 5 μg/mL, and after washing, detected sFasL bound to Fas using polyclonal anti-FasL antibodies. It was found that the binding curve of mut-sFasL was shifted to the right, indicating decreased binding ability for Fas. It is possible that the decreased binding ability could contribute to the lack of competition between the mut-sFasL and WT-sFasL.

To further examine the activity of mut sFasL at the Fas receptor, it was tested whether mut-sFasL would inhibit an antibody capable of binding and activating Fas in viable cells. Caspase 3/7 activity in Jurkat cells following 5-hour incubation with the Fas-activating antibody CH11, a control antibody, or different molar ratios of mut-sFasL and antibody, was measured. The mut-sFasL did not attenuate the caspase 3/7 activation induced by CH11, and in fact, at very high molar ratios it increased CH11 activity (FIG. 3B). This experiment further demonstrates that that mut-sFasL has low binding affinity for Fas, and that the inhibitory mechanism is likely unrelated to direct antagonism at the receptor level.

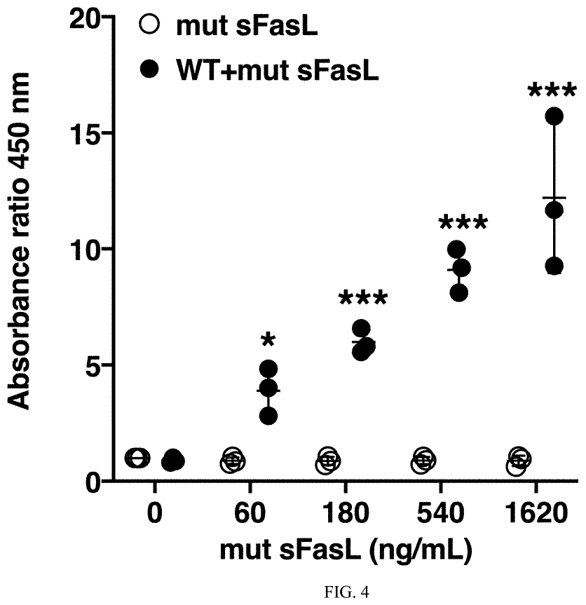

The mut-sFasL forms complexes with WT sFasL. In order to be active, sFasL must form trimers or higher order multimers. Therefore, it was tested whether the mut-sFasL forms complexes with WT-sFasL. FLAG-tagged mut-sFasL was mixed with biotinylated WT-sFasL at the same molar ratios used in the experiments shown in FIG. 1. The mixture was incubated in streptavidin-coated well to capture the biotinylated WT-sFasL. After washing unbound material, FLAG was detected using an anti-FLAG antibody, which would detect FLAG-tagged mut-sFasL complexed with biotinylated WT-sFasL. As shown in FIG. 4, the FLAG-tagged mut-sFasL by itself did not bind to the plate, even at high concentrations. However, there was a dose-dependent increase in FLAG signal in wells treated with fixed concentrations of biotinylated WT sFasL and increasing concentrations of FLAG-tagged mut sFasL, showing the formation of complexes.

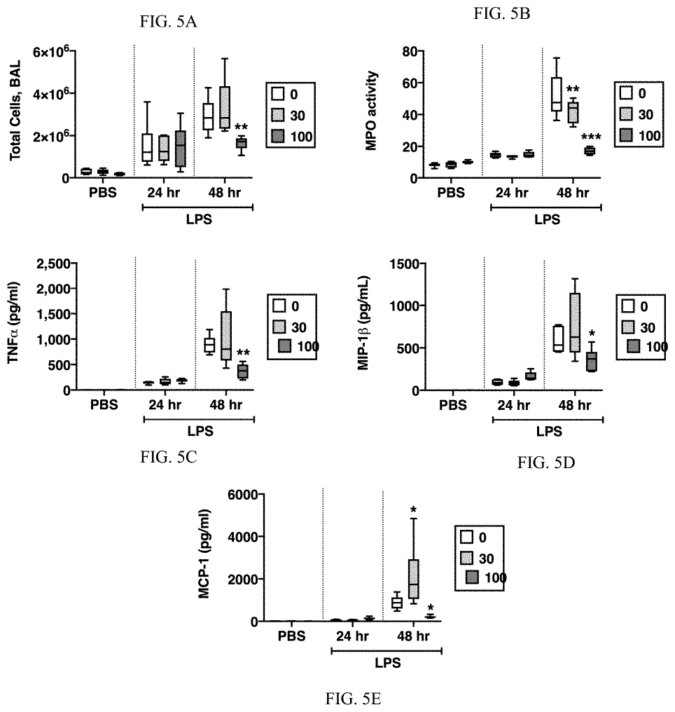

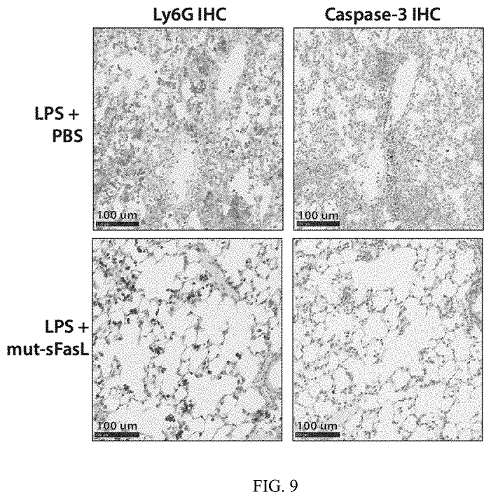

The mut-sFasL attenuates the lung inflammatory response to intratracheal LPS. The next step was to determine whether the mut-sFasL would affect the response of mice to bacterial lipopolysaccharide (LPS), an established model of sterile lung injury. Mice received intratracheal instillations of LPS, 2.5 μg/g, or PBS (“Time=0”). An “early group” of mice received mut-sFasL after 4 hours and were euthanized at 24 hours. A “late group” received mut-s-FasL at 24 hours followed by euthanasia at 48 hours. It was found that in the late group, mut-sFasL led to mild changes in some measures of the inflammatory response (FIG. 5). Specifically, in the late group the BAL total cell counts were decreased in mice receiving 100 ng/mL mut-sFasL (FIG. 5A). Total lung MPO activity, a measure of total lung neutrophils, also was significantly decreased in the mut-sFasL-treated mice at doses of 30 and 100 ng/mL (FIG. 5B). The attenuation of the neutrophil response was associated with a decrease in the BAL concentrations of TNF-α, MIP-1 β and MCP-1 (FIGS. 5C-E). The neutrophil chemoattractants KC and MIP-2 were not affected, and there was an increase in IL-1(3 and MIP-la (Table 2). Representative images of immunohistochemical staining for LyG (neutrophil marker) and caspase 3 are shown in FIG. 9.

TABLE 2
BAL cytokine concentration.

| | PBS | | | 24 hr | | | 48 hr | | |
|---|---|---|---|---|---|---|---|---|---|
| | 0 | 30 | 100 | 0 | 30 | 100 | 0 | 30 | 100 |
| IL-1β | 0.2 ± 0.0 | 0.2 ± 0.0 | 0.2 ± 0.1 | 11.3 ± 3.4 | 14.3 ± 10.6 | 18.6 ± 5.5 | 24.0 ± 7.4 | 37.8 ± 23.3 | 44.5 ± 23.1 |
| MIP-1α | 0.1 ± 0.05 | 0.08 ± 0.03 | 0.29 ± 0.28 | 54.3 ± 14.4 | 47.9 ± 10.6 | 85.3 ± 20.0 | 109.3 ± 19.4 | 154.5 ± 78.7 | 153.9 ± 42.4 |
| KC | 1.5 ± 1.1 | 1.2 ± 0.6 | 1.9 ± 0.9 | 152.6 ± 32.7 | 118.4 ± 43.7 | 154.4 ± 94.3 | 110.4 ± 44.5 | 113.2 ± 55.8 | 136.9 ± 51.9 |
| MIP-2 | 0.1 ± 0.0 | 0.1 ± 0.0 | 2.3 ± 2.8 | 107.7 ± 23.2 | 78.0 ± 28.1 | 124.1 ± 29.1 | 107.5 ± 14.8 | 146.7 ± 72.7 | 135.7 ± 29.9 |

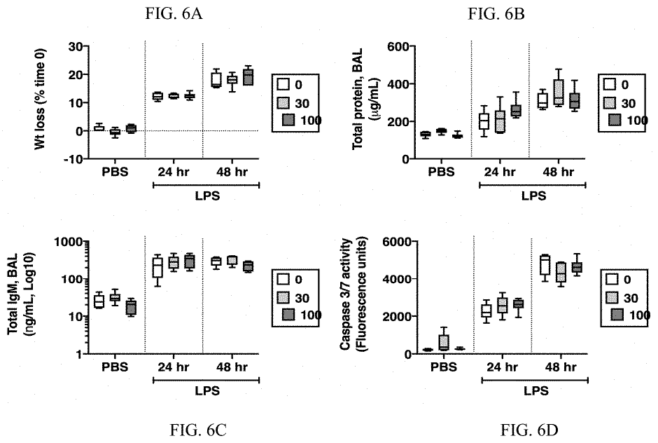

The LPS-treated animals lost weight, but the extent of weight loss was not affected by the mut-sFasL (FIG. 6A). The mut-sFasL did not lead to an increase in lung apoptosis or permeability measurements in the PBS-treated mice, and it did not affect the LPS-induced increases in lung homogenate caspase-3/7 activity (used as marker for apoptotic activity) or BAL concentrations of total protein and the high molecular weight protein IgM (used as measurements of alveolar barrier permeability) (FIGS. 6B-D).

Discussion. It was demonstrated, in separate gain-of-function and loss-of-function studies, that Fas activation is associated with lung injury in mice, and these studies that have been corroborated by other groups (Del Sorbo L, et al. Crit Care Med 44: e604-613, 2016; Gil S, et al. Respir Res 13: 91, 2012; Matute-Bello G, et al. J Infect Dis 191: 596-606, 2005; Matute-Bello G, et al. Am J Physiol Lung Cell Mol Physiol 281: L328-L335, 2001; Matute-Bello G, et al. Am J Pathol 158: 153-161, 2001; and Weckbach S, et al. J Trauma Acute Care Surg 74: 792-800, 2013). It has also been shown that the main target cells of sFasL in lung injury are alveolar epithelial cells, which respond to Fas ligation with both apoptosis and cytokine release (Matute-Bello G, et al. J Immunol 175: 4069-4075, 2005; Matute-Bello G, et al. Am J Pathol 158: 153-161, 2001; and Nakamura M, et al. Am J Pathol 164: 1949-1958, 2004). The relevance of these findings was demonstrated in studies showing that in humans, sFasL can exist in a long-form, containing a 26-amino acid “stalk” at the N-terminus, and a short form, in which this stalk has been cleaved by metalloproteinases (Herrero R, et al. J Clin Invest. 2011; 121(3):1174-90); importantly, the long form is active, and during ARDS, oxidation of important residues of the stalk region prevent its cleavage, this stabilizing the active “long” form (Herrero R, et al. J Clin Invest 121: 1174-1190, 2011). The results described herein show that mutation of the charged residues of the sFasL stalk region into non-charged alanines results in a mutant protein that has attenuated activity, and importantly, acts as an inhibitor of the WT protein in vitro, and retains mild inhibitory properties in vivo.

The Fas/FasL system is composed of Fas (CD95) and its cognate ligand, FasL (CD178). In the lungs, Fas is expressed ubiquitously (Matute-Bello G, et al. Am J Physiol Lung Cell Mol Physiol 281: L328-L335, 2001), whereas FasL is expressed in lung epithelial cells (Wadsworth S J, et al. J Allergy Clin Immunol 126: 366-374, 374 e361-368, 2010), neutrophils, macrophages, and lymphocytes (Beck J M, et al. Infect Immun 77: 1053-1060, 2009). Fas is a type I transmembrane receptor member of the TNF superfamily of proteins (Itoh N, et al. Cell 66: 233-243, 1991). The intra-cytoplasmic domain of Fas can exist in two forms, a “closed” form and an “open” form (Scott F L, et al. Nature 457: 1019-1022, 2009). The open form of Fas binds the adapter protein FADD via death domain (DD) homotypic interactions, however, the open form is unstable in the absence of FasL. Binding of Fas to multimers of FasL stabilizes Fas to the open form by inducing clustering of Fas in tetramers or larger oligomers (Scott F L, et al. Nature 457: 1019-1022, 2009). Once FADD binds to Fas, a signaling cascade is initiated, leading to activation of the cysteine protease caspase 8, activation of caspase 3, and execution of apoptosis. Thus, three important events in the initiation of Fas signaling are, first, multimerization of FasL, second, binding of FasL to Fas; third, clustering of the Fas/FasL complex and stabilization of the open form of Fas.

Fas ligand is a 281-amino acid, 40 kDa type II transmembrane protein. The N-terminal region is cytoplasmic and the C-terminal domain is extracellular. The extracellular domain is divided in two additional domains: a 144-amino acid TNF-homology domain (THD), which mediates receptor binding and is rich in aromatic and hydrophobic residues, and a 34 amino acid stalk region that links the THD with the transmembrane domain (Berg D, et al. Cell Death Differ 14: 2021-2034, 2007). The extracellular domain can be shed by a number of proteases including ADAM10, MMP3 and MMP7, resulting in sFasL (Kirkin V, et al. Cell Death Differ 14: 1678-1687, 2007; Schulte M, et al. Cell Death Differ 14: 1040-1049, 2007; Vargo-Gogola T, et al. Arch Biochem Biophys 408: 155-161, 2002; and Wadsworth S J, et al. J Allergy Clin Immunol 126: 366-374, 374 e361-368, 2010). However, the biological role of sFasL has been controversial. Early studies suggested that sFasL lacked bioactivity and in fact could even function as an inhibitor of membrane-bound FasL (Schneider P, et al. J Exp Med 187: 1205-1213, 1998; Suda T, et al. J Exp Med 186: 2045-2050, 1997; and Tanaka M, et al. Nat Med 4: 31-36, 1998). O'Reilly et al. suggested that sFasL retains pro-inflammatory activity, but not pro-apoptotic activity (O'Reilly L, et al. Nature 461: 659-663, 2009). In contrast, Shudo et al. suggested the opposite (Shudo K, et al. Eur J Immunol 31: 2504-2511, 2001). Some studies suggested that the stalk region of sFasL may be involved to its bioactivity (Suda T, et al. J Immunol 157: 3918-3924, 1996), and it is certainly involved in sFasL oligomerization (Berg D, et al. Cell Death Differ 14: 2021-2034, 2007). It was found that the bioactivity of sFasL depends of the length of its stalk region, which in vivo exists in two forms: an inactive short form, containing amino acids 129-281, and a bioactive long form, containing amino acids 103-281 (Herrero R, et al. J Clin Invest 121: 1174-1190, 2011). The long form predominates in the BAL of patients with ARDS, is stabilized by oxidation, and it accounts for most of its pro-apoptotic activity.

The role of the stalk region of sFasL on sFasL activity was further investigated. Interestingly, sFasL binds Fas at the C-terminal side of the protein, suggesting that the mechanism behind the difference in activity between the long and short forms could be a conformational change in the tertiary structure interfering with one of the three steps of Fas signaling described herein (multimerization, binding or receptor clustering). Thus, 8 charged residues of the stalk region were changed to the non-charged amino acid alanine in order to reduce ionic binding of the stalk region to putative binding partners. The resultant mutant protein (mut-sFasL) had significantly attenuated activity compared to WT sFasL and acted as an inhibitor of the WT protein. Interestingly, the prediction model of this mutation suggests that changing the charged residues to alanine results in conformational changes in the stalk region of the trimeric form of the sFasL (FIG. 7B). This computer modeling of sFasL and of the mutant sFasL has excellent concordance with the predicted structure of “short” sFasL described in a study characterizing Fas-FasL interactions (Schneider P, et al. J Biol Chem.1997; 272(30):18827-33). In this work, the authors noted that the “short” sFasL trimers did not bind Fas in a stable manner in the absence of glycosylation. Based on these observations and the computer model in which the spatial arrangement of the stalk region is altered, the stalk region may play a role in glycosylation and Fas interaction.

The results disclosed here showed that the inhibition was unlikely a result of direct competition for the receptor or increased binding affinity. The observation that the mut-sFasL did not affect the activity of a Fas-binding antibody further suggested that the inhibitory mechanism was unlikely at the receptor level, and may involve mut-sFasL interaction with the WT sFasL protein prior to receptor engagement. Capture of the mut-sFasL by WT sFasL protein provided additional supportive evidence that mut-sFasL forms heteromultimeric complexes with WT sFasL. We speculate the inhibitory action of mut-sFasL is not by direct antagonism of the receptor. Rather, sFasL heterodimerization with the mutant protein is likely responsible for the loss of activity of WT sFasL. Confirmation of the inhibitory mechanism will require further studies to elucidate the precise structure of these complexes and their interactions with Fas.

Some experiments in this report were conducted using Jurak cells as a known responder of Fas-FasL pathway. However, the studies described herein focused on the effect of sFasL on airway epithelium. The functional effect of activating the Fas pathway in the murine epithelial cell line La4 is known (Herrero R, et al. Am J Physiol Lung Cell Mol Physiol. 2013; 305(5):L377-88). In that study, it was demonstrated the activation of the Fas signaling pathway by the activating antibody Jo2 increased cell death and LA4 monolayer permeability in a dose dependent manner. Furthermore, inhibition of caspase activity by the pan-caspase inhibitor zVAD reversed cell death and monolayer permeability induced by Jo2, implicating caspase activity in the functional consequences of Fas activation. These findings have been further confirmed and elaborated in human pulmonary alveolar epithelial cells (HPAEpiC) (Herrero R, et al. Thorax. 2019; 74(1):69-82). HPAEpiC exposed to the long form of sFasL induced caspase 3, with subsequent increases in permeability of HPAEpiC monolayers followed by cell death. It was further shown that tight junction proteins ZO-1 and occludin were down-regulated with Fas activation. Importantly, pretreatment of HPAEpiCs with caspase 3-specific inhibitor zDEVD.fmk reversed the effect of sFasL on HPAEpiC monolayer permeability and ZO-1/occludin expression, demonstrating the functional effect is mediated through caspase 3. Described herein is evidence demonstrating the inhibitory effect of mut sFasL on caspase 3/7 induction in epithelial cells. WT:mut sFasL molar ratios of 1:27 and 1:81 significantly reduced caspase 3/7 activity as well as Annexin V translocation on SAECs in this study.

Given that the variant sFasL attenuated WT-sFasL activity in vitro, next it was tested whether it could attenuate lung injury in vivo. There are several lines of evidence suggesting that targeting the Fas/FasL system could be of particular importance as a potential therapeutic for acute lung injury, because it affects multiple pathways leading to two of the important features of lung injury: disruption of the alveolar/epithelial barrier and inflammation. Disruption of the alveolar epithelial barrier occurs by at least two mechanisms: First, although Fas is ubiquitously expressed in the lungs, adoptive transfer studies and macrophage depletion studies have confirmed that Fas activation specifically targets non-myeloid cells, leading to caspase 3/7 activation, apoptosis and loss of membrane integrity (Bem R A, et al. Am J Physiol Lung Cell Mol Physiol 295: L314-325, 2008; Matute-Bello G, et al. J Immunol 175: 4069-4075, 2005; Perl M, et al. Am J Pathol 167: 1545-1559, 2005). In addition to this structural injury, Fas activation also affects the expression of alveolar epithelial tight junction proteins, a caspase-dependent effect that occurs even in the absence of apoptosis (Herrero R, et al. Thorax 74: 69-82, 2019). Combined, both mechanisms lead to impairment of the epithelial barrier, as well as eversible disruption of alveolar fluid clearance (Herrero R, et al. Am J Physiol Lung Cell Mol Physiol 305: L377-388, 2013). In addition to its effects on alveolar epithelial barrier, Fas activation is also a powerful inducer of pro-inflammatory cytokine release, by a mechanism involving activation of the MAP-kinase pathway and transcription factors such as AP-1 (Matute-Bello G, et al. J Immunol. 1999; 163:2217-25; and Matute-Bello G, et al. J Immunol. 2005; 175(6):4069-75). In vitro, this effect is seen in myeloid cells and in alveolar epithelial cells, but in vivo, the presence of Fas in the alveolar epithelium is required for lung inflammation by sFasL (Farnand A W, et al. Am J Respir Cell Mol Biol 45: 650-658, 2011). Importantly, studies using sterile and non-sterile models of injury and Fas-deficient animals suggest that the contribution of the Fas/FasL system to lung inflammation is not trivial (Del Sorbo L, et al. Crit Care Med 44: e604-613, 2016; Gil S, et al. Respir Res 13: 91, 2012; and Matute-Bello G, et al. J Infect Dis 191: 596-606, 2005). Thus, inhibition of the Fas/FasL system may play an important role as a novel therapeutic strategy for lung injury.

A sterile model of lung injury induced by bacterial lipopolysaccharide as a stereotypical, one-hit model was chosen that would allow evaluation of one-time administration of the mut-sFasL. The mutant protein was administered either 4 hours after LPS, to test its effects during the very early phases of lung injury, or 24 hours after LPS, to evaluate its effects at a point when lung injury is fully established. While early administration had no measurable effect, in the late group, mut-sFasL reduced some parameters of inflammation. The dominant effect was a reduction in total BAL cells and whole lung MPO (a measurement of the total lung content of neutrophils), as well as the cytokines TNF-α, MCP-1 and MIP-1β (CCL4). Taken together, the 24 hr data show a reduction in lung inflammation, with reduced TNFα and MCP-1 and a reduction in lung neutrophil content. Surprisingly, the main murine neutrophil chemoattractants KC (CXCL1) and MIP-2 (CXCL2) were not affected. This pattern of attenuated neutrophilic response with unchanged concentrations of KC and MIP-2 and no change in permeability markers is very similar to that seen in Fas-deficient lpr mice exposed to LPS and mechanical ventilation (Gil S, et al. Respir Res. 2012; 13:91). In that study, it was found that the lack of functional Fas was associated with decreased deposition of anti-KC:KC immune complexes in the lungs, which are known to enhance the inflammatory response (Krupa A, et al. Am J Respir Cell Mol Biol. 2007; 37(5):532-43; and Fudala R, et al. Clin Sci (Lond). 2010; 118(8):519-26). The data provided herein support that inhibition of the Fas/FasL system by a sFasL with mutated stalk region is not injurious in normal lungs and can have a biological effect in injured lungs in vivo, reproducing the phenotype of Fas-deficient lpr mice in sterile lung injury.

In summary, the results described herein demonstrated that mutation of the charged amino acids of the stalk region of sFasL to neutral markedly attenuates the activity of sFasL. Importantly, the mutated protein heterodimerizes with wild type sFasL, and inhibits its activity in vitro. The mutated protein did not have important pro-injury or pro-inflammatory effects at any dose when administered 24 hours after intratracheal LPS, confirming the important role of the stalk region in the bioactivity of sFasL in vivo. In addition, the mut-sFasL was a weak inhibitor of the inflammatory response. The results described herein a focus on alterations of the stalk region and support the development of inhibitors that bind to the stalk region. It was concluded that changes in the structure of the stalk region of sFasL interfere with its function and may be the basis of therapeutic agents against the Fas/FasL system.

1. A composition comprising a peptide comprising the amino acid sequence QLFX₁LQX₂X₃LAX₄LX₅X₆STSQMX₇TASSLX₈K, wherein X₁ is H or a non-polar amino acid residue;
X2 is K or a non-polar amino acid residue;
X3 is E or a non-polar amino acid residue;
X4 is E or a non-polar amino acid residue;
X5 is R or a non-polar amino acid residue;
X6 is E or a non-polar amino acid residue;
X7 is H or a non-polar amino acid residue; and
X8 is E or a non-polar amino acid residue.

2. The composition of claim 1, wherein at least one of X₁-X₈ is a non-polar amino acid residue.

3. (canceled)

4. The composition of claim 1, wherein each of X₁-X₈ is a non-polar amino acid residue.

5. (canceled)

6. A composition comprising a peptide, wherein the peptide comprises the amino acid sequence of SEQ ID NO: 1: QLFHLQKELAELRESTSQMHTASSLEK, wherein one or more of the charged amino acids in SEQ ID NO: 1 is substituted for a non-polar amino acid residue.

7. The composition of claim 6, wherein the non-polar amino acid residue is an alanine residue, a glycine residue, a proline residue, a valine residue, a leucine residue, an isoleucine residue, a methionine residue or a tryptophan residue.

8.-10. (canceled)

11. The composition of claim 6, wherein the peptide comprises the amino acid sequence QLFALQAALAALAASTSQMATASSLAK (SEQ ID NO: 2).

12. The composition of claim 6, wherein the peptide comprises the amino acid sequence QLFALQKELAELRESTSQMHTASSLEK (SEQ ID NO: 3).

13. The composition of claim 6, wherein the peptide comprises the amino acid sequence QLFHLQAELAELRESTSQMHTASSLEK (SEQ ID NO: 4).

14. The composition of claim 6, wherein the peptide comprises the amino acid sequence QLFHLQKALAELRESTSQMHTASSLEK (SEQ ID NO: 5).

15. The composition of claim 6, wherein the peptide comprises the amino acid sequence QLFHLQKELAALRESTSQMHTASSLEK (SEQ ID NO: 6).

16. The composition of claim 6, wherein the peptide comprises the amino acid sequence QLFHLQKELAELAESTSQMHTASSLEKK (SEQ ID NO: 7).

17. The composition of claim 6, wherein the peptide comprises the amino acid sequence QLFHLQKELAELRASTSQMHTASSLEK (SEQ ID NO: 8).

18. The composition of claim 6, wherein the peptide comprises the amino acid sequence QLFHLQKELAELRESTSQMATASSLEK (SEQ ID NO: 9).

19. The composition of claim 6, wherein the peptide comprises the amino acid sequence QLFHLQKELAELRESTSQMHTASSLAK (SEQ ID NO: 10).

20. The composition of claim 2, wherein the polypeptide has at least 80% sequence identity to any of SEQ ID NOs: 1-10.

21.-23. (canceled)

24. The composition of claim 1, further comprising a pharmaceutically acceptable carrier.

25. (canceled)

26. A method of treating a pulmonary disease, the method comprising: administering to a subject with pulmonary disease a therapeutically effective amount of a composition of claim 1.

27. (canceled)

28. The method of claim 26, wherein the pulmonary disease is obstructive pulmonary disease (COPD), emphysema, asthma, idiopathic pulmonary fibrosis, pneumonia, tuberculosis, cystic fibrosis, bronchitis, pulmonary hypertension, interstitial lung disease, acute respiratory distress syndrome (ARDS), acute lung injury (ALI), idiopathic pulmonary pneumonitides or lung cancer.

29. (canceled)

30. (canceled)

31. A method of inhibiting sFasL activity in a subject, the method comprising administering a therapeutically effective amount of a composition of claim 1 to a subject in need thereof.

32. (canceled)

33. (canceled)

34. The method of claim 31, wherein the subject has a pulmonary disease, wherein the pulmonary disease is obstructive pulmonary disease (COPD), emphysema, asthma, idiopathic pulmonary fibrosis, pneumonia, tuberculosis, cystic fibrosis, bronchitis, pulmonary hypertension, interstitial lung disease, acute respiratory distress syndrome (ARDS), acute lung injury (ALI), idiopathic pulmonary pneumonitides or lung cancer.

35. (canceled)

36. (canceled)


SEQUENCE LISTING (12 sequences)

SEQ ID NO: 1 - protein, 27 aa, Artificial Sequence
  Synthetic construct
QLFHLQKELAELRESTSQMHTASSLEK

SEQ ID NO: 2 - protein, 27 aa, Artificial Sequence
  Synthetic construct
QLFALQAALAALAASTSQMATASSLAK

SEQ ID NO: 3 - protein, 27 aa, Artificial Sequence
  Synthetic construct
QLFALQKELAELRESTSQMHTASSLEK

SEQ ID NO: 4 - protein, 27 aa, Artificial Sequence
  Synthetic construct
QLFHLQAELAELRESTSQMHTASSLEK

SEQ ID NO: 5 - protein, 27 aa, Artificial Sequence
  Synthetic construct
QLFHLQKALAELRESTSQMHTASSLEK

SEQ ID NO: 6 - protein, 27 aa, Artificial Sequence
  Synthetic construct
QLFHLQKELAALRESTSQMHTASSLEK

SEQ ID NO: 7 - protein, 28 aa, Artificial Sequence
  Synthetic construct
QLFHLQKELAELAESTSQMHTASSLEKK

SEQ ID NO: 8 - protein, 27 aa, Artificial Sequence
  Syntheic construct
QLFHLQKELAELRASTSQMHTASSLEK

SEQ ID NO: 9 - protein, 27 aa, Artificial Sequence
  Synthetic construct
QLFHLQKELAELRESTSQMATASSLEK

SEQ ID NO: 10 - protein, 27 aa, Artificial Sequence
  Synthetic construct
QLFHLQKELAELRESTSQMHTASSLAK

SEQ ID NO: 11 - protein, 178 aa, Artificial Sequence
  Synthetic construct
QLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPSHKVYMRNSKYPQDLVMMEGKMMSYCTTGQMWARSSYLGAVFNLTSADHLYVNVSELSLVNFEESQTFFGLYKL

SEQ ID NO: 12 - protein, 27 aa, Artificial Sequence
  Synthetic construct
  MISC_FEATURE (4)..(4): H or a non-polar amino acid residue
  MISC_FEATURE (7)..(7): K or a non-polar amino acid residue
  MISC_FEATURE (8)..(8): E or a non-polar amino acid residue
  MISC_FEATURE (11)..(11): E or a non-polar amino acid residue
  MISC_FEATURE (13)..(13): R or a non-polar amino acid residue
  MISC_FEATURE (14)..(14): E or a non-polar amino acid residue
  MISC_FEATURE (20)..(20): H or a non-polar amino acid residue
  MISC_FEATURE (26)..(26): E or a non-polar amino acid residue
QLFXLQXXLAXLXXSTSQMXTASSLXK